(19) United States
(12) Patent Application Publication
(10) Pub. No.: US 2024/0156591 A1
(43) Pub. Date: May 16, 2024

(54) LOW-PROFILE PROSTHETIC HEMI HEART VALVE DEVICES AND METHODS FOR USE

(71) Applicant: Sutra Medical, Inc., Lake Forest, CA (US)

(72) Inventors: Thuy Pham, Lake Forest, CA (US); Caitlin Martin, Foothilll Ranch, CA (US); Tongran Qin, Lake Forest, CA (US); Wei Sun, Foothill Ranch, CA (US)

(21) Appl. No.: 18/389,558

(22) Filed: Nov. 14, 2023

Related U.S. Application Data
(60) Provisional application No. 63/425,650, filed on Nov. 15, 2022.

(51) Int. Cl. A61F 2/24

(52) U.S. Cl. CPC A61F 2/2418; A61F 2/90

(57) ABSTRACT

A low-profile prosthetic hemi valve for treatment of a regurgitant heart valve. In one example, the prosthetic valve includes a large upper portion for anchoring in a dilated annulus, and a smaller open lower portion that is suspended in the blood flow path near the native leaflet coaptation line when implanted. The prosthetic valve includes at least one flexible prosthetic leaflet that moves between an open configuration to a closed position along with native leaflets of the valve. In operation, the at least one prosthetic leaflet serves as a conformable coaptation surface for at least one native leaflet, and the posterior side of the lower portion of the frame serves as a coaptation surface for at least one native leaflet to completely seal the native valve orifice. Optionally, the frame can comprise an open flow channel to enhance diastolic filling of the ventricle.

### CROSS REFERENCE TO RELATED APPLICATIONS

The present application claims benefit of co-pending U.S. provisional application Ser. No. 63/425,650, filed Nov. 15, 2022, and is related to U.S. Publication Nos. 2021/0212824 and 2017/0258589, the entire disclosures of which are expressly incorporated by reference herein.

### TECHNICAL FIELD

The present application relates generally to replacement heart valves, for example, for replacing diseased mitral and/or tricuspid valves. More particularly, the present application relates to tissue-based, collapsible and expandable replacement heart valves and to systems and methods for implanting such valves.

### BACKGROUND

The mitral valve (MV) has two distinct large leaflet cusps, or leaflets. As shown in FIG. 1A, the MV is on the left side of the heart and located between the left atrium and the left ventricle. Referring to FIG. 1B, the mitral valve apparatus consists of a mitral annulus, two leaflets, chordae tendineae (“chords”), two papillary muscles and the left ventricular myocardium. The mitral annulus is subdivided into an anterior portion and a posterior portion. Normally, the anterior mitral leaflet is connected to the aortic valve via the aortic-mitral curtain, and the posterior mitral leaflet is hinged on the posterior mitral annulus. The chords originate from either the two major papillary muscles or from multiple small muscle bundles attaching to the ventricular wall and connect to the free edge of the mitral leaflets. Chords are composed mainly of collagen bundles, which give the chords high stiffness and maintain minimal extension to prevent the leaflets from billowing into the left atrium during systole.

When the mitral valve is closed, the respective anterior and posterior leaflets are in close contact to form a single zone of apposition. As one skilled in the art will appreciate, normal mitral valve function involves a proper force balance, with each of its components working congruently during a cardiac cycle. Pathological alterations affecting any of the components of the mitral valve, such as chord rupture, annulus dilatation, papillary muscle displacement, leaflet calcification, and myxomatous disease, can lead to altered mitral valve function and cause mitral valve regurgitation (MR).

Mitral regurgitation, shown in FIG. 2, is dysfunction of the mitral valve that causes an abnormal leakage of blood from the left ventricle back into the left atrium during systole (i.e., the expulsion phase of the heart cycle in which blood moves from the left ventricle into the aorta). While trivial mitral regurgitation can be present in healthy patients, moderate to severe mitral regurgitation is one of the most prevalent forms of heart valve disease. The most common causes of mitral regurgitation include ischemic heart diseases, non-ischemic heart diseases, and valve degeneration. Both ischemic (mainly due to coronary artery diseases) and non-ischemic (idiopathic dilated cardiomyopathy for example) heart diseases can cause functional, or secondary, mitral regurgitation through various mechanisms, including impaired left ventricle wall motion, left ventricle dilatation, and papillary muscle displacement and dysfunction. In functional mitral regurgitation, the mitral valve apparatus remains normal. Incomplete coaptation of the leaflets is due to enlargement of the mitral annulus secondary to left ventricle dilation and possibly left atrium enlargement. In addition, patients with functional mitral regurgitation can exhibit papillary muscle displacement due to the left ventricle enlargement, which results in excessive tethering of the leaflets. In contrast, degenerative (or organic) mitral regurgitation is caused by structural abnormalities of the mitral leaflets and/or the subvalvular apparatus, which can include stretching or rupture of tendinous chords.

The current treatments for mitral valve diseases include surgical repair and replacement of the mitral valve. Mitral valve repair, benefiting from improved understanding of mitral valve mechanics and function, may be now preferred to complete mitral valve replacement. However, the complex physiology and three-dimensional anatomy of the mitral valve and its surrounding structure present substantial challenges when performing these repair procedures.

In one early example of a transcatheter mitral valve replacement device, Endovalve-Herrmann (Micro Interventional Devices, Inc.) developed a mitral prosthesis that had a foldable Nitinol-based valve with a sealing skirt. Similarly, Tendyne Holdings, Inc. produces a prosthetic mitral valve replacement device comprising a pericardial valve with a self-expandable Nitinol stent. The device is designed for transapical delivery and has a ventricular fixing anchor. CardiAQ uses a pericardial valve with a Nitinol self-expandable stent in their mitral valve replacement device. Finally, Tiara (Neovasc, Inc.) uses a mitral valve replacement system that is deliverable trans-apically with a 30 Fr catheter that has anchor structures, and a pericardial valve on a self-expandable stent with a D-shaped atrial portion and a ventricular portion that has an outer coating. These devices and the techniques to deliver the mitral prosthesis into the operative position are still at development stages and, though promising, challenges to the efficacy of these devices continue to exist.

The noted challenges to an efficacious mitral valve replacement device generally include operative delivery challenges; positioning and fixation challenges; seal and paravalvular leakage challenges; and hemodynamic function challenges such as left ventricular outflow tract (LVOT) obstruction and possible mitral stenosis. With respect to the noted operative delivery challenges, since a conventional mitral prosthetic is larger than a conventional aortic prosthesis, it is more difficult to fold and compress the larger mitral prosthesis into a catheter for deployment as well as retrieval through either conventional trans-apical or trans-femoral delivery techniques.

Turning to the positioning and fixation challenges, instability and migration are the most prominent obstacles given that the mitral valve is subjected to high and repetitive loads in a cardiac cycle, with a high transvalvular pressure gradient that is near zero at diastole and can rise to 120 mmHg or more during systole and higher than 150 mmHg of systolic pressure for patients with aortic stenosis and systemic hypertension. The lack of calcium distribution at the mitral annulus also affects device stability and anchoring. Further, the transcatheter mitral valve replacement can be easily dislodged as the heart moves during each beating cycle.

With respect to sealing and paravalvular leakage, good sealing between the native annulus and the prosthesis that minimizes paravalvular leak is desirable. Conventionally, a prosthetic mitral valve is smaller than the diseased native valve and additional material is added around the prosthetic valve to compensate for the large native mitral annulus. Undesirably, adding more material to a prosthetic valve increases the size of the delivery system. Further, this can create an elevated forward flow pressure gradient across the valve or even stenosis.

Finally, with respect to the preservation of hemodynamic function, the operative positioning of a prosthetic mitral valve, which is conventionally large as described above, should not obstruct the mitral orifice during diastole or the LVOT at the anterior portion of the mitral annulus during systole.

Posterior mitral valve repair techniques such as Polares and Half-moon consist of a non-mobile posterior coaptation surface for the native anterior mitral valve and thus avoid LVOT obstruction.

Accordingly, it would be beneficial to have a heart valve leaflet replacement system that does not suffer from the shortcomings and deficiencies of conventional valve prosthetics. For example, it may be desirable to secure the prosthetic mitral valve replacement system to the native mitral annulus. It may also be desirable to improve positioning of a mitral prosthesis and prevent leaking of blood between the mitral prosthesis and the native mitral valve without creating stenosis. Similarly, it may be desirable to prevent further dilation of the native mitral annulus. Furthermore, other desirable features and characteristics will become apparent from the subsequent detailed description and the appended claims, taken in conjunction with the accompanying drawings and the foregoing technical field and background.

### SUMMARY

The present application is directed generally to prosthetic heart valves and methods for implanting prosthetic heart valve, and, more particularly, to low-profile prosthetic hemi heart valves or prosthetic hemi-valves configured to fill a regurgitant orifice area during systole to treat mitral regurgitation without restricting diastolic filling of the left ventricle. It is contemplated that the low-profile prosthetic hemi-valves herein can be implanted via an open surgical procedure or percutaneously via catheter. For clarity, it will be appreciated that, although this disclosure may focus on the treatment of functional mitral regurgitation, it is contemplated that the low-profile prosthetic hemi-valves and the associated methods can be used or otherwise configured to be used to treat other valve disease conditions such as degenerative mitral regurgitation and regurgitation of other valves (e.g., tricuspid valve) of the human heart, or could be used or otherwise configured to be used in other mammals suffering from valve deficiencies as well.

In one aspect, the low-profile prosthetic hemi-valve is configurable or otherwise sizable to be crimped down to fit within a delivery catheter and to subsequently be selectively re-expanded to an operative size and position once removed from the delivery catheter within the heart. In a further aspect, the low-profile prosthetic hemi-valve can comprise a stent, with an open lower ventricular portion attached to a radially flared out upper portion, where an angled neck portion forms a transition between the upper and lower portions of the hemi-valve.

In one aspect, the upper portion can be configured to facilitate anchoring of the stent, which can help prevent dislodgement of the stent. The lower portion can be suspended in the blood flow tract and house at least one flexible prosthetic leaflet. In another aspect, the prosthetic hemi-valve can comprise a sealing skirt that can be coupled to at least a portion of the inner and/or outer surfaces of the stent.

In one exemplary aspect, at least one prosthetic leaflet can be mounted on the inner lumen of the stent and/or on at least a portion of the outer side of the stent. The at least one flexible prosthetic leaflet can be configured to be mobile throughout the cardiac cycle and coapt with at least one native valve leaflet. The lower portion of the stent frame can also be configured as a coaptation surface for one or more native heart valve leaflets.

In one aspect, the upper portion of the low-profile prosthetic hemi-valve is configured with a large diameter and flared out shape for annular anchoring and atrial apposition. The lower portion of the low-profile prosthetic hemi-valve has a fish mouth-shape, resembling the healthy native mitral valve coaptation line. An angled neck region forms a transition between the upper and lower portions of the hemi-valve.

In one example, the low-profile prosthetic hemi-valve device can comprise a partial elliptical, upper stent portion that is configured for posterior mitral annulus anchoring from one trigone to the other. The lower portion of the frame can comprise a smaller fish mouth-shape with a larger major axis corresponding to the commissure-to-commissure anatomical direction and a smaller minor axis corresponding to the anterior-to-posterior anatomical direction of the mitral annulus. In this example, the lower portion is attached to the upper portion such that it is suspended in the blood flow path near the native mitral valve coaptation line in the operative position. At least one flexible prosthetic leaflet can be attached to the inner surface of the lower portion of the frame, wherein it is configured to be mobile throughout the cardiac cycle, and coapt with at least a portion of the native anterior mitral leaflet. Further in this example, the native posterior and commissure mitral leaflets are undisturbed and can move normally and coapt with the outer surface of the lower portion of the frame, such that during systole, the entire mitral orifice is sealed closed. During diastole, the blood flow can push the at least one prosthetic leaflet towards the inner surface of the lower portion of the frame, such that flow between the device and the native anterior mitral leaflet is uninhibited. Further in this example, the frame can comprise at least one flow channel in the upper and/or neck portion of the frame to promote diastolic flow between the native posterior mitral leaflet and the outer surface of the lower portion of the frame.

In another example, the upper stent portion can be configured as a full ring to be anchored on the mitral annulus.

Someone skilled in the art can appreciate that, in some circumstances, it is desirable to have a large upper stent portion to facilitate device anchoring because many patients with mitral regurgitation have a large, dilated mitral annulus, but a smaller lower portion supporting the at least one prosthetic leaflet, such that the device can be more easily crimped into a low-profile diameter for safer transcatheter delivery and implantation. Furthermore, a smaller structure within the native valve and ventricle may be desirable because it has minimal impact on the surrounding native tissues. In the case of functional mitral regurgitation, the mitral valve apparatus is often functional; the regurgitant orifice is the result of dilation of the heart. The native leaflets can no longer fully coapt with each other but can still help with sealing around the implant. The at least one flexible prosthetic leaflet can cover the regurgitant orifice. Smaller prosthetic leaflets are also beneficial for implant durability given their smaller surface area, they experience less load.

In some circumstances, it may be desirable for the prosthetic valve to have an effective orifice area similar to the native valve. Otherwise, the patient could experience an elevated pressure gradient or stenosis across the replacement valve. Someone skilled in the art can appreciate that the effective orifice area of the mitral valve is naturally reduced by implantation of a prosthetic valve with a smaller diameter than the native annulus. The at least one flow channel through the frame in this example, can significantly increase the effective orifice area of the prosthetic hemi-valve and help maintain normal function of the native mitral leaflets.

In one example, the minor axis of the lower portion of the frame is smaller than that of the upper portion. The major axis of the lower portion of the frame is the same or similar to the minimum major axis dimension of the upper portion of the frame. In this example, the lower portion of the frame follows a shallower curve compared to the upper portion. Thus, the neck portion at the minor axis of the device extends radially inward from the upper portion to the lower portion of the frame. The neck at the major axis of the device is nearly vertical.

In one aspect, at least one prosthetic leaflet can be mounted to the inner surface of the lower portion of the stent frame. The prosthetic leaflet can be configured to be flexible and mobile throughout the cardiac cycle. During the systolic phase, the at least one prosthetic leaflet extends to coapt with native anterior leaflets by extending radially outwards from the lower portion of the frame to prevent leakage between the anterior surface of the implant and the native anterior leaflet, while the native posterior leaflets extend to coapt with the outer surface of the lower portion of the frame to prevent leakage between the posterior surface of the implant and the native posterior leaflet. During diastole, the at least one prosthetic leaflet is configured to move towards the lower portion of the frame to allow for blood to flow from the left atrium to the left ventricle between the anterior surface of the implant and the native anterior leaflet while the at least one flow channel in the neck region of the frame allows blood to flow from the left atrium to the left ventricle between the posterior surface of the implant and the native posterior leaflet.

In one example, the at least one prosthetic leaflet can mimic the configuration of the native mitral posterior leaflets with three adjoined semilunar cusps on the lower portion of the stent.

In one aspect, the sealing skirt material can be made of polymers, fabric, biological tissue, and the like. The skirt can be a single piece of material, or alternatively, the skirt can be configured from multiple separate pieces of material, coupled to the frame via non-absorbable suture or string. It is contemplated that the skirt material can be configured to promote tissue in-growth at the annulus, and protect against abrasion between the frame and surrounding anatomic structures.

In one aspect, delivery of the low-profile prosthetic hemi-valve can be conducted using several desired delivery access approaches, such as, for example and not meant to be limited to, a surgical approach, a trans-septal approach, a trans-atrial, or a trans-apical approach. In one exemplary aspect, the trans-septal approach can comprise creating an opening in the internal jugular or femoral vein for the subsequent minimally invasive delivery of portions of the low-profile prosthetic hemi-valve through the superior vena cava, which flows into the right atrium of the heart. In this exemplary aspect, the access path of the trans-septal approach crosses the atrial septum of the heart, and once achieved, the components of the prosthetic hemi-valve can operatively be positioned in the left atrium, the native mitral valve, and the left ventricle. In one aspect, it is contemplated that a main delivery catheter can be placed therein the access path to allow desired components of the prosthetic hemi-valve to be operatively positioned in the left atrium without complications.

It can be appreciated by an individual skilled in the art that due to the nature of the implant that is designed to fill a regurgitant orifice between native leaflets versus an entire native leaflet and/or valve, it naturally has a smaller profile, compared to a full valve or large coaptation surface, allowing a greater portion of the at-risk population to undergo a transcatheter mitral valve replacement operation.

In one aspect, a plurality of dual guiding and fixation members can be operatively positioned and implanted at desired locations in the native annulus prior to the delivery of the replacement prosthetic hemi-valve. In this aspect, the dual guiding and fixation members can improve the subsequent positioning and anchoring of the replacement prosthetic hemi-valve.

Various implementations described in the present application can include additional systems, methods, features, and advantages, which can not necessarily be expressly disclosed herein but will be apparent to one of ordinary skill in the art upon examination of the following detailed description and accompanying drawings. It is intended that all such systems, methods, features, and advantages be included within the present application and protected by the accompanying claims.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1A is a surgeon's view schematic of the healthy native mitral valve during systole. FIG. 1A shows that the mitral leaflets coapt to form a “fish-mouth” coaptation line. There are two mitral valve leaflets: the anterior leaflet and the posterior leaflet. The posterior leaflet comprises three adjoined semi-lunar shapes.

FIG. 1B is a long axis cross-sectional view schematic of the native mitral valve leaflets coapting during systole.

FIGS. 2A-2H illustrate the mitral valve during systole in the mitral regurgitation disease state. FIG. 2A is a surgeon's view schematic of the mitral valve with Type I regurgitation where the native leaflets move normally. FIG. 2B is a long-axis cross-sectional view of the heart with Type I mitral regurgitation. FIG. 2C is a surgeon's view schematic of Type II regurgitation where there is increased motion of one or more native leaflets. FIG. 2D is a long-axis cross-sectional view of the heart with Type II mitral regurgitation. FIG. 2E is a schematic of Type Ma regurgitation where there is restricted motion of one or more of the native leaflets during systole and diastole. FIG. 2F is a long-axis cross-sectional view of the heart with Type Ma mitral regurgitation. FIG. 2G is a surgeon's view schematic of Type Mb regurgitation where there is restricted motion of one or more native leaflets during systole. FIG. 2H is a long-axis cross-sectional view of the heart with Type IIIb mitral regurgitation.

FIGS. 3A and 3B depict an example of a low-profile prosthetic hemi-valve device for the treatment of mitral regurgitation with a stent frame, three prosthetic leaflets, a sealing skirt, and a posterior flow channel created by a window in the skirt. FIG. 3A shows the device from the front view. FIG. 3B shows the device from the side view.

FIG. 4A is a schematic view of an exemplary aspect of a low-profile prosthetic hemi-valve device with a flared out upper stent portion configured for annular anchoring and a smaller fish mouth-shaped lower portion. Three prosthetic leaflets are mounted on the inner surface of the lower portion of the frame. A fabric skirt material covers the upper portion of the frame that will be in contact with the annulus in operation. There is a window through the skirt in the neck portion of the frame that will be positioned over the mitral orifice in operation to allow for a flow channel through the device.

FIG. 4B is a surgeon's view illustration of the low-profile hemi-valve device of FIG. 4A implanted in a native mitral valve during systole. In this view, the native anterior mitral leaflet can be seen extended out radially towards the prosthetic leaflets. The prosthetic leaflets conform to the coaptation zone of the anterior leaflet.

FIG. 4C is a long axis cross-section schematic of the low-profile prosthetic hemi-valve device of FIGS. 4A and 4B during systole. In this view, the native anterior mitral leaflet can be seen coapting with the prosthetic leaflet mounted on the inner surface of the lower portion of the frame, and the posterior mitral leaflet can be seen coapting with the posterior side of the lower portion of the frame.

FIG. 5A is a surgeon's view schematic of a low-profile prosthetic hemi-valve implanted in a mitral valve during diastole. The native mitral leaflets have moved outward radially and the prosthetic leaflets have moved towards the lower portion of the frame allowing blood to flow from the left atrium to the left ventricle between the anterior side of the prosthetic hemi-valve and the native anterior leaflet as well as between the posterior side of the prosthetic hemi-valve and the native posterior leaflet through the posterior flow channel.

FIG. 5B is a long axis cross section view schematic of the low-profile prosthetic hemi-valve of FIG. 5A implanted in a mitral valve during diastole.

FIGS. 6A and 6B show a schematic of an exemplary dual guiding and fixation (DGF) mechanism to stabilize the low-profile prosthetic hemi-valve on the native annulus during operation. FIG. 6A depicts the DGF member embodiment with a screw-like anchor to engage the tissue, a locking mechanism to engage the low-profile prosthetic hemi-valve frame, and a trailing tail to guide deployment of the device. FIG. 6B is a cross-sectional schematic of deployment of the low-profile prosthetic hemi-valve device guided by the tail of the previously implanted anchor.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The devices and methods herein can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, and, as such, can, of course, vary. It is also to be understood that the terminology used herein is for the purpose of describing particular aspects only and is not intended to be limiting.

The following description is provided as an enabling teaching of various aspects and examples of the invention. To this end, those skilled in the relevant art will recognize and appreciate that many changes can be made to the various aspects and examples described herein, while still obtaining the beneficial results of the devices and methods described herein. It will also be apparent that some of the desired benefits of the devices and methods herein can be obtained by selecting some of the features of the described examples without utilizing other features.

Accordingly, those who work in the art will recognize that many modifications and adaptations to the devices and methods herein are possible and can even be desirable in certain circumstances and are a part of the present invention. Thus, the following description is provided as illustrative of the principles of the present invention and not in limitation thereof.

For clarity, it will be appreciated that this disclosure will focus on the treatment of functional mitral regurgitation, however it is contemplated that the heart valve leaflet replacement system and the associated methods can be used or otherwise configured to be used to treat other types of mitral regurgitation or to replace other diseased valves of the human heart, such as tricuspid valve, or could be used or otherwise configured to be used in other mammals suffering from valve deficiencies as well.

As used throughout, the singular forms “a,” “an” and “the” include plural referents unless the context clearly dictates otherwise. Thus, for example, reference to “a leaflet” can include two or more such leaflets unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance can or cannot occur, and that the description includes instances where said event or circumstance occurs and instances where it does not.

The word “or” as used herein means any one member of a particular list and also includes any combination of members of that list. Further, one should note that conditional language, such as, among others, “can,” “could,” “might,” or “may,” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular embodiment.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these cannot be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems can be understood more readily by reference to the following detailed description of preferred embodiments.

Throughout the description, the terms “prosthetic valve” and “prosthesis” and “valve stent” and “heart valve leaflet replacement device” and “valve device” are used interchangeably and is contemplated as a heart valve replacement device described herein.

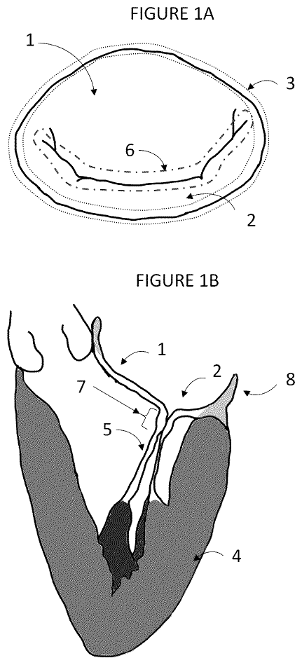

Referring to FIG. 1A and FIG. 1B, the mitral valve consists of anterior 1 and posterior 2 leaflets, originating from the annulus 3 and extending into the left ventricle 4. In a healthy patient, the anterior mitral leaflet 1 and the posterior mitral leaflet 2 come together and coapt during systole to prevent backward flow through the mitral valve. The mitral coaptation line 6 has a characteristic “fish-mouth” shape shown in FIG. 1A. Referring to FIG. 1B, chordae tendinae 5 hold the free edges of the leaflets down in the ventricle such that they can coapt during systole with a coaptation zone 7 of approximately 1 to 5 mm and block blood from flowing from the left ventricle 4 to the left atrium 8.

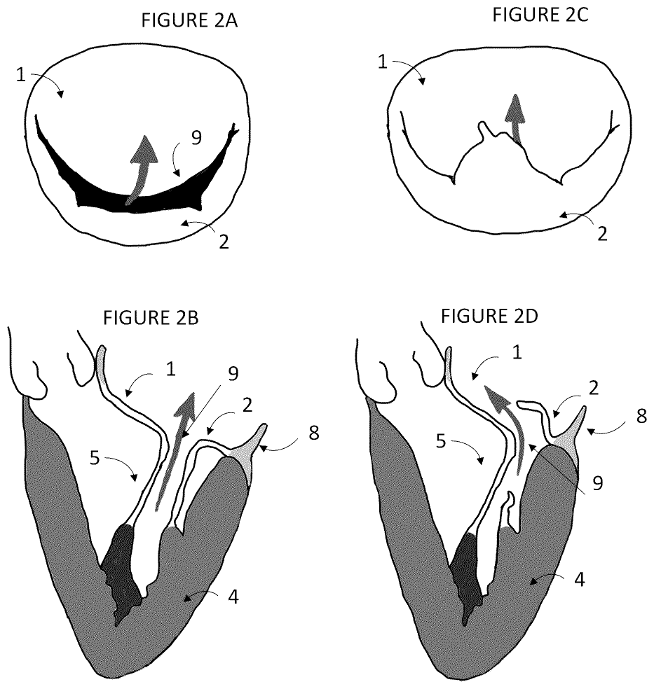

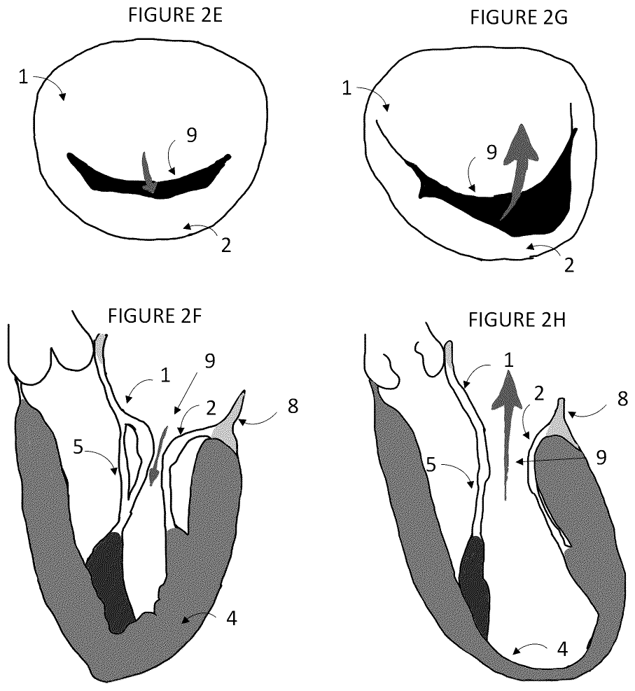

In the mitral regurgitation diseased state, e.g., depicted in FIGS. 2A-2H, the native coaptation zone 7 is lost or incomplete such there is a gap or regurgitant orifice 9 in the valve during systole leading to the leakage of blood from the left ventricle 4 back to the left atrium 8 during systole. Mitral regurgitation can occur with normal mitral leaflet motion due to dilation of the heart, e.g., as depicted in FIG. 2A and FIG. 2B. Regurgitation can occur due to excessive native leaflet motion resulting from a ruptured chord, e.g., as shown in FIG. 2C and FIG. 2D. Regurgitation can also occur due to restricted leaflet motion during systole, e.g., shown in FIG. 2G and FIG. 2H, or during both diastole and systole, e.g., shown in FIG. 2E and FIG. 2F.

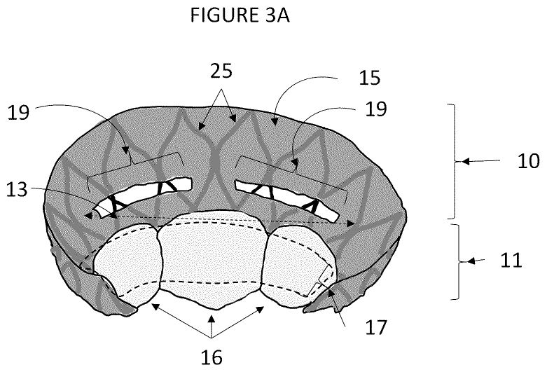

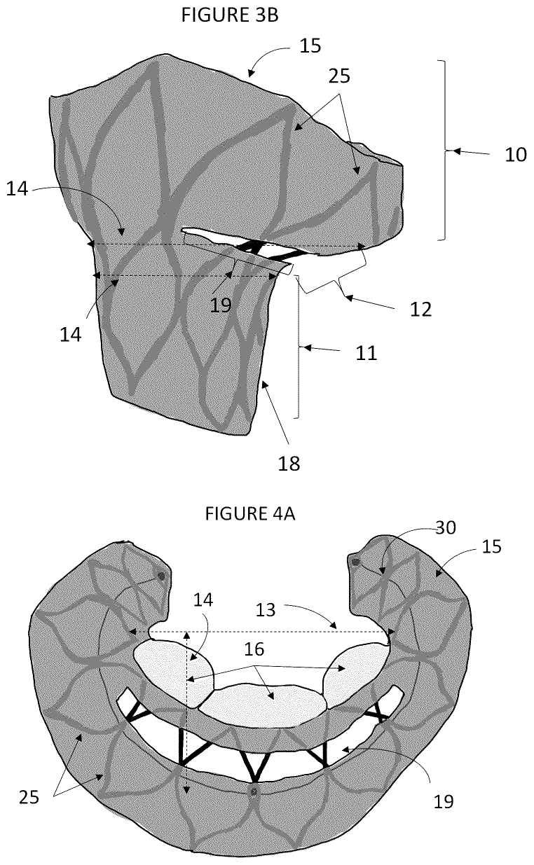

The low-profile prosthetic hemi-valve devices, systems, and methods presented herein may be used to treat mitral regurgitation in patients with normal native mitral leaflets and chordae by filling the regurgitant orifice 9 during systole. FIG. 3 depicts an example of a low-profile prosthetic hemi-valve device. The low-profile prosthetic hemi-valve can comprise a stent frame with a plurality of diamond shaped cells 25, with an open lower ventricular portion 11 attached to a radially flared out upper portion 10, where an angled neck portion 12 forms a transition between the upper 10 and lower portions 11 of the device. Portions of the frame can be covered in a sealing skirt 15. At least one flexible and mobile prosthetic leaflet 16 can be mounted on the lower portion of the frame 11. The low-profile prosthetic hemi-valve device can comprise an upper portion 10 that is configured for mitral annulus 3 anchoring. In operation, the neck portion 12 will be within the native valve annulus, and the lower portion 11 will be suspended in the left ventricle 4.

It is contemplated that the device has a major axis 13 and minor axis 14 at the neck portion of the frame, wherein the major axis 13 corresponds to the commissure-to-commissure anatomical direction and the minor axis 14 corresponds to the anterior-to-posterior anatomical direction of the mitral annulus. In one example, the upper portion of the frame 10 has a larger minor axis 14, i.e., greater than 5 mm larger, than the lower portion of the frame 11, and a similar or larger major axis 13 compared to that of the lower portion of the frame 11. The lower portion of the frame 11 is configured to be open. The upper portion of the frame can be configured in an open or closed shape.

The prosthetic leaflet 16 can be configured to be flexible and mobile throughout the cardiac cycle. During the systolic phase, the at least one prosthetic leaflet 16 extends to coapt with native anterior leaflets 1 by extending radially outwards from the lower portion of the frame to prevent leakage between the anterior coaptation surface 17 of the implant and the native anterior leaflet 1, while the native posterior leaflets 2 extend to coapt with the outer surface of the lower portion of the frame 11 to prevent leakage between the posterior coaptation surface 18 of the implant and the native posterior leaflet 2. During diastole, the at least one prosthetic leaflet 16 is configured to move towards the lower portion of the frame 11 to allow for blood to flow from the left atrium 8 to the left ventricle 4 between the anterior coaptation surface of the implant 17 and the native anterior leaflet 1. Additionally, the neck portion 12 can be configured with a posterior flow channel 19 that allows blood to flow from the left atrium to the left ventricle between the posterior coaptation surface of the implant 18 and the native posterior leaflet 2.

FIG. 3A shows an example of the device from the front view with a sealing skirt and three prosthetic leaflets 16 comprising an anterior coaptation surface 17 and a posterior flow channel 19 created by a plurality of windows in the sealing skirt 15. FIG. 3B shows the device from the side view where the posterior coaptation surface 18 and the difference in the minor axis 14 dimension between the upper 10 and lower 11 frame portions can be better appreciated. In this example, the upper portion of the frame 10 is configured to open towards the anterior mitral leaflet 1 and to be anchored along the posterior annulus from the medial trigone to the lateral trigone. Further the lower portion of the frame 11 is configured to open towards the anterior mitral leaflet 1 and to be suspended in the mitral orifice spanning from the medial commissure to the lateral commissure.

It is contemplated that the prosthetic leaflet 16 can be comprised of a thin, flexible material including human or animal tissues, polymer, or fabric.

It is contemplated the upper 10, lower 11, and neck portion 12 of the frame can be comprised of a metallic material such as one or more of Nitinol, cobalt chromium, or stainless steel, or a polymer material.

It is contemplated that the sealing skirt 15 can be comprised of a thin, flexible material including one or more of human or animal tissues, polymer, or fabric.

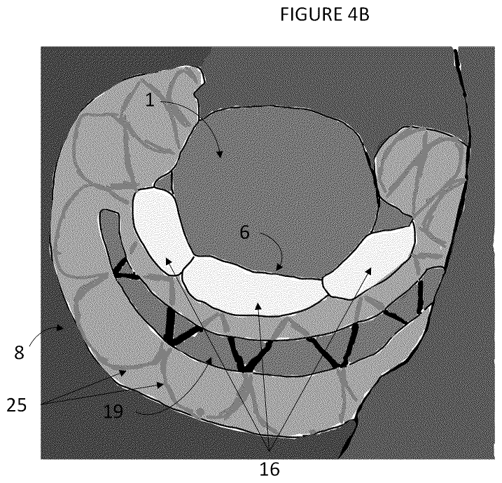

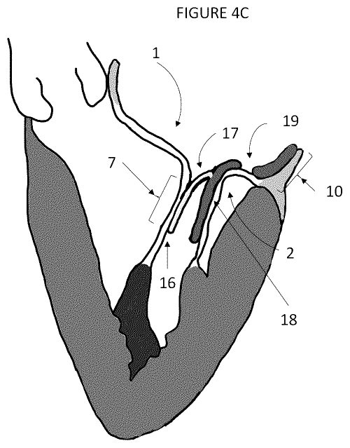

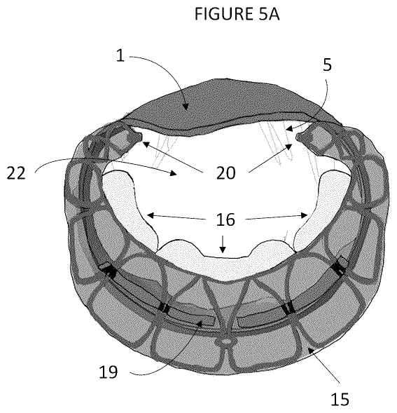

It is contemplated that the upper portion of the frame 10 can be configured to facilitate anchoring of the device on the native annulus 3. In operation, the upper portion 10 can be anchored to the native annulus 3, such that the lower portion 11 is suspended in the left ventricle 4 and is parallel to the blood flow. In one example for the treatment of mitral regurgitation, the lower portion of the frame 11 can be configured with a fish mouth-shape similar to the native coaptation line 6. The at least one prosthetic leaflet 16 can be configured as three distinct cusps similar to the native posterior leaflet as shown in FIG. 4A. FIG. 4B is a schematic of the low-profile prosthetic hemi-valve embodiment of FIG. 4A implanted in a heart during systole. In operation, the prosthetic leaflet 16 can extend radially from the inner surface of the lower portion of the frame 11 during systole creating an anterior coaptation surface 17 to coapt with the native anterior leaflet 1. It can be appreciated that the open nature of the lower portion of the frame allows for the native anterior leaflet 1 to extend radially within the confines of the low-profile prosthetic hemi-valve device frame such that it can coapt with the anterior coaptation surface 17 of the device. The device can re-create the characteristic fish-mouth coaptation line 6 seen in the healthy native mitral valve as shown in FIG. 4B. The flexible nature of the prosthetic leaflet 16 allows it to conform to the native anterior leaflet 1 to seal the mitral orifice. The native posterior leaflet 2 can be seen through the posterior flow channel 19 of the device coapting with the posterior coaptation surface 18 of the device on the posterior side of the lower portion of the frame 11. FIG. 4C is a long axis cross-sectional schematic of the low-profile prosthetic hemi-valve implanted in a heart during systole. The prosthetic leaflet 16 functions as a conformable anterior coaptation surface 17 for the native anterior leaflet 1 to contact, and the posterior side of the lower portion of the frame 11 serves as a posterior coaptation surface 18 for the native posterior leaflet 2 to contact. The native anterior 1 and posterior 2 leaflets together with the prosthetic leaflet 16, seal the entire mitral orifice.

It is contemplated that the posterior coaptation surface 18 can be wrapped in or made by a soft, thin material such as human or animal tissues, polymer, or fabric to prevent the native posterior leaflet 2 from becoming damaged over time. It is further contemplated that the posterior coaptation surface 18 can be comprised of at least one prosthetic leaflet 16, or mobile surface, to coapt with the native posterior leaflet 2.

Referring again to FIG. 4B, the flexible nature of the prosthetic leaflet 16 allows it to substantially change shape, compared to the resting shape in FIG. 4A, to conform to the native anterior leaflet 1 during systole. One skilled in the art can appreciate that because the device relies on the native anterior leaflet 1 along with the prosthetic leaflet 16 to seal the mitral orifice, the durability of the implant relies on the long-term function of both the native anterior leaflet 1 and the prosthetic leaflet 16. Accordingly, it is desirable to impose minimal damage to the anterior leaflet 1 in operation. By having a flexible prosthetic leaflet 16 to coapt with a flexible native leaflet, impact damage is minimized. The smaller prosthetic leaflet 16 in the low-profile hemi valve design will also have improved durability compared to a larger leaflet due to the lower surface area.

Referring again to FIG. 4A, it is contemplated that the sealing skirt material can be coupled to the upper portion of the frame along the anchoring line 30 that will be in contact with the native annulus in operation. Optionally, the sealing skirt can comprise a high-surface area to volume ratio material such that it can be easily crimped and can promote tissue ingrowth.

It is contemplated that the upper portion of the frame 10 can be configured as either a closed shape or an open shape. One knowledgeable in the art can appreciate that because the upper portion of the frame will sit on the left atrial 8 side of the annulus in operation, it should not impinge or hinder the native leaflet function. It is further contemplated that the upper portion of the frame 10 can be configured with a full or partial elliptical-, circular-, or D-shape to better conform to the annulus 3 and left atrium 8.

It is contemplated that the lower portion of the frame 11 could be configured with various other open shapes such as a partial elliptical-, circular-, or parabolic-shape to aid in the treatment of valvular regurgitation. For instance, it is contemplated that the lower portion of the frame 11 could be configured with a more V-shape to simulate the coaptation line between the anterior and posterior leaflets with the septal leaflet in the tricuspid valve to treat tricuspid regurgitation. It is also contemplated that the lower portion of the frame 11 could be configured with various heights along the long-axis direction. For instance, it is contemplated that the lower portion of the frame 11 could be configured with shorter vertical heights at the two sides of the open frame to avoid contact with the papillary muscle heads in the left ventricle.

It is contemplated that the lower portion of the frame 11 can be configured with a major axis 13 similar or slightly larger than that of the native annulus such that it is wide enough to allow for a native leaflet to fit inside the confines of the lower portion of the frame 11 to coapt with the prosthetic leaflet 16. Further, it is contemplated that the lower portion of the frame 11 can be configured with a minor axis 14 that is smaller than that of the native annulus such that the overall device footprint inside the left ventricle as well as the crimped profile is desirably low.

It can be appreciated by those skilled in the art, that it is desirable to have a device that can anchor well, reduce mitral regurgitation without negatively impacting the surrounding anatomical structures and functions, and be able to fit in a low-profile delivery catheter for improved patient safety. The upper portion 10 of the device presented in this disclosure has a large diameter for anchoring on a dilated mitral annulus 3, yet minimal material such that it can be easily crimped to a small diameter. The lower portion 11 of the device has more material, including a plurality of prosthetic leaflets 16, but a smaller size, particularly the minor axis 14, such that it can also be crimped to a small diameter.

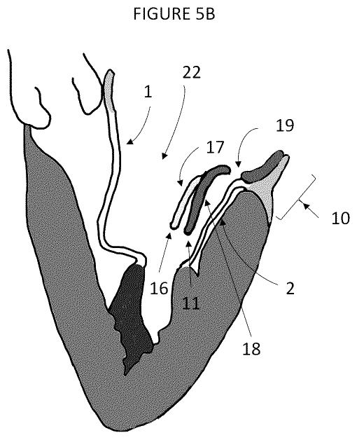

FIGS. 5A and 5B show schematics of an example of the low-profile prosthetic hemi-valve device implanted in a mitral valve during diastole. The surgeon's view schematic in FIG. 5A shows that the native anterior 1 and posterior 2 leaflets move outward radially towards the left ventricle 4 wall, and the prosthetic leaflet 16 moves towards the lower portion of the frame 11 during diastole. Thus, there are two blood flow channels through the mitral orifice: one between the native anterior leaflet 1 and the anterior coaptation surface 17 of the device, namely the anterior flow channel 22, and one through the posterior flow channel 19 of the device created by the plurality of windows in the sealing skirt 15. Together the two diastolic blood flow channels create a large effective orifice area which is desirable for heart function. FIG. 5B shows the long axis cross-section view of the device in the mitral valve during diastole. This view shows that the posterior flow channel 19 is positioned above the gap between the native posterior leaflet 2 and the posterior coaptation surface 18 of the device.

It is contemplated that the low-profile prosthetic hemi-valve device can be configured with a plurality of posterior flow channels 19 of varying sizes and shapes or optionally without any posterior flow channel 19 such that there is only one blood flow channel through the mitral orifice, the anterior flow channel 22. The flexible and movable prosthetic leaflet 16 can be configured such that the anterior coaptation surface 17 moves substantially away from the native anterior leaflet 1 during diastole to allow ample flow from the left atrium 8 to the left ventricle 4.

It is contemplated that the frame can be configured with a plurality of diamond shaped cells 25 such that the frame can be selectively crimped and loaded into a small diameter catheter. In the preferred embodiment, the upper portion of the frame 10 can span the posterior annulus from the medial trigone to the lateral trigone in a “D-shape” designed to conform with the mitral annulus and left atrial wall 8. Further in this example, the lower portion of the frame can span the mitral valve coaptation line 6 from the medial commissure to the lateral commissure in a “fish-mouth” shape similar to the native mitral valve coaptation line 6. The exposed tips of the diamond cells in the frame can optionally be bent inwards radially to avoid interference with surrounding tissues including the left atrium 8, native posterior leaflet 2, chordae tendinae 5, and native anterior leaflet 1.

The upper portion of the frame 10 can optionally be configured with a plurality of eyelets for engaging an anchoring mechanism, a crimping mechanism, and/or a loading mechanism into a catheter for transcatheter delivery in a patient.

The lower portion of the frame 11 can optionally be configured with a plurality of eyelets to facilitate crimping and/or loading the crimped the device into a catheter for transcatheter delivery in a patient.

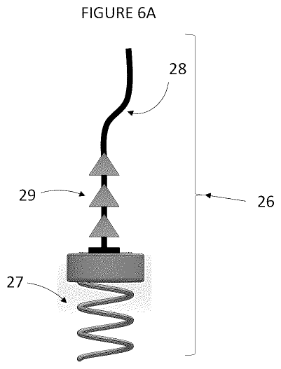

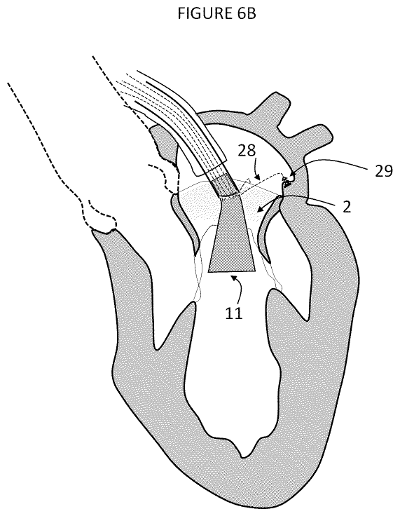

In an exemplary method, the low-profile prosthetic hemi-valve device can be guided and fixed into place on the annulus via a plurality of dual-guiding-fixation (DGF) members 26 shown in FIG. 6A and FIG. 6B. Additional information regarding DGF members and systems and methods for delivering them may be found in U.S. Publication No. 2021/0212824, the entire disclosure of which is expressly incorporated by reference herein.

Referring to FIG. 6A, the DGF member 26 can comprise a screw-like anchor 27 configured to engage the annular tissue at the distal end, and a long trailing tail member 28, on the proximal end. Referring to FIG. 6B, in operation, the plurality of DGF member anchors 27 can be implanted in the annulus tissue first. The trailing tail members 28 can then be passed through eyelets in the upper portion of the frame 10 to guide the deployment of the low-profile prosthetic hemi-valve device to the annulus 3. Optionally, an additional locking mechanism 29 can then be passed over the trailing tail 28 to sandwich the upper portion of the frame 10 against the implanted anchor 27. The DGF member tail can be configured to be selectively removable once the implant is secured in place.

It should be emphasized that the above-described aspects are merely possible examples of implementation, merely set forth a clear understanding of the principles of the present disclosure. Many variations and modifications can be made to the above-described embodiment(s) without departing substantially from the spirit and principles of the present disclosure. All such modifications and variations are intended to be included herein within the scope of the present disclosure, and all possible claims to individual aspects or combinations of elements or steps are intended to be supported by the present disclosure. Moreover, although specific terms are employed herein, as well as in the claims which follow, they are used only in a generic and descriptive sense, and not for the purposes of limiting the described invention, nor the claims which follow.

What is claimed is:

1. A heart valve repair device, comprising:
a stent frame with an upper portion configured for annular anchoring, an open lower portion configured to be suspended in a blood flow path of a native valve within a heart, and a neck transition portion between the upper portion and the lower portion, wherein a minor axis of the lower portion is smaller than a minor axis of the upper portion;
at least one flexible prosthetic leaflet mounted on the lower portion of the frame that moves from an open position to a closed position through normal function of the heart; and
a sealing skirt attached to at least one portion of the frame.

2. The heart valve repair device of claim 1, wherein the stent frame comprises a network of cells configured to be radially collapsible and can be expandable in an operative position.

3. The heart valve repair device of claim 2, wherein the upper portion comprises a plurality of cells comprising free stent cell tips configured to be curved inward radially with respect to the rest of the cells.

4. The heart valve repair device of claim 2, wherein the lower portion comprises a plurality of cells comprising free stent cell tips configured to be curved inward radially with respect to the rest of the cells.

5. The heart valve repair device of claim 1, wherein the upper portion comprises a plurality of through-holes.

6. The heart valve repair device of claim 1, wherein the lower portion comprises a plurality of through-holes.

7. The heart valve repair device of claim 1, wherein the at least one prosthetic leaflet comprises three leaflets.

8. The heart valve repair device of claim 1, wherein the stent frame is configured with at least one open flow channel.

9. The heart valve repair device of claim 5, wherein the plurality of through-holes are configured to accept passage of a plurality of dual guiding and fixation member tails and locking members.

10. The heart valve repair device of claim 1, wherein the stent frame has a partial elliptical shape between the upper portion and the lower portion.

11. The heart valve repair device of claim 1, wherein the upper portion has a flared out shape for annular anchoring and atrial apposition and the lower portion has a fish mouth-shape, resembling the healthy native mitral valve coaptation line.

12. The heart valve repair device of claim 1, wherein the neck region forms a transition between the upper portion and the lower portion.

13. The heart valve repair device of claim 1, wherein the upper portion has a partial elliptical shape configured for posterior mitral annulus anchoring from one trigone to the other, and the lower portion comprise a fish mouth-shape with a larger major axis dimension corresponding to a commissure-to-commissure anatomical direction of the native valve and a smaller minor axis dimension corresponding to an anterior-to-posterior anatomical direction of the native valve.

14. The heart valve repair device of claim 13, wherein the minor axis dimension of the lower portion of the frame is smaller than a minor axis dimension of the upper portion, and wherein the major axis dimension of the lower portion is the same or similar to a major axis dimension of the upper portion.

15. The heart valve repair device of claim 14, wherein the lower portion follows a shallower curve compared to the upper portion and, the neck portion at the minor axis dimension of the device extends radially inward from the upper portion to the lower portion.

16. The heart valve repair device of claim 1, wherein the sealing skirt covers both the upper portion and the lower portion.

17. A method for implanting the prosthetic hemi valve device of claim 1, comprising:
introducing the stent frame into a patient's heart adjacent a native valve annulus; and
anchoring the upper portion to the native valve annulus such that the lower portion is suspended in the blood flow tract of the native valve.

18. The method of claim 17, wherein the lower portion provides a coaptation surface for one or more native heart valve leaflets of the native valve.
